(19) United States
(12) Patent Application Publication
(10) Pub. No.: US 2008/0254507 A1
(43) Pub. Date: Oct. 16, 2008

(54) Anti-Hiv Drug, Polypeptide Constituting the Same, Gene Encoding the Polypeptide and Method of Producing the Anti-Hiv Drug

(75) Inventors: Haruo Tanaka, Tokyo (JP); Junji Inokoshi, Saitama (JP); Satoshi Omura, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: KIIM Pharm. Lab, Inc., Fukushima (JP)

(21) Appl. No.: 11/791,862

(22) PCT Filed: Nov. 30, 2005
(86) PCT No.: PCT/JP05/21981

§ 371 Date: Jan. 22, 2008

(30) Foreign Application Priority Data
Nov. 30, 2004 (JP) 2004-346644

(51) Int. Cl. C12P 21/00; C07K 14/00; C07H 21/00; C12N 1/21

(52) U.S. Cl. 435/69.1; 530/350; 536/23.1; 435/252.33; 435/252.3

(57) ABSTRACT

It is intended to provide an anti-HIV drug characterized by containing multimeric actinohivin, a polypeptide which is multimeric actinohivin; a gene encoding the same; and a method of producing the anti-HIV drug. The anti-HIV drug inhibits the synctium formation and has an enhanced effect

### FIELD OF THE INVENTION

The present invention relates to an anti-viral agent for human immunodeficiency virus (HIV), i.e., an anti-HIV agent, a polypeptide constituting the same, DNA encoding the polypeptide, a transformant transformed with the DNA, and a method for producing the anti-HIV agent. More specifically, the present invention relates to an anti-viral agent for HIV-1 (an anti-HIV agent), a polypeptide constituting the same, DNA encoding the polypeptide, a transformant transformed with the DNA, and a method for producing the anti-HIV agent.

### BACKGROUND ART

Currently, reverse transcriptase inhibitors and protease inhibitors are used as therapeutic agents for treating acquired immunodeficiency syndrome (AIDS) caused by HIV.

Reverse transcriptase inhibitors are classified into two major groups: nucleotide-type inhibitors and non nucleotide-type inhibitors. It is known that prolonged administration of nucleotide-type inhibitors will cause severe side effects, and that resistant strains will emerge after about one year from the initial administration of the inhibitors. Non nucleotide-type inhibitors will cause fewer side effects, but due to their highly specific action, resistant strains will emerge early on. On the other hand, protease inhibitors show good anti-viral activities even when administered alone, but they will generally be effective only transiently, and the sensitivities will decline due to the mutations of the amino acid sequences of HIV proteases. In addition, they involve problems of low stabilities in vivo, side effects such as the digestive system disorder and so on.

The infection of HIV is established by its adhering and entering to a host cell via the binding between an HIV surface protein, glycoprotein 120 (gp120), and a surface receptor of the host cell, CD4. It is expected that a substance that inhibits the binding of gp120 and CD4 will inhibit the adhesion of HIV and the cell, and thus it will prevent HIV infection. Thus, attempts have been made to inhibit the binding between gp120 and CD4. For example, attempts such as the administration of soluble CD4 prepared by genetic engineering to human subjects have been done. However, by such a method, an anti-HIV activity was not observed due to the reasons such as short half-lives in vivo even though the inhibitory activity had been recognized in vitro. Further, it was revealed that murine cells carrying expressed human CD4 could not be infected, suggesting the presence of a second adhesion molecule.

Recently, it was repeatedly reported that the second receptor was a member of chemokine receptors. HIV strains are divided into two major groups, i.e., one containing macrophage-tropic strains (M-tropic HIV) and the other containing T cell-tropic strains (T-tropic HIV). It was suggested that the difference in cell tropism observed between these groups of viral strains was due to the differences in the molecular species of the second receptor. That is, it was demonstrated that the cell tropism of HIV strains was determined by the type of the second receptor molecule, either CC chemokine receptor 5 (CCR5: M-tropic HIV receptor) or CXC chemokine receptor 4 (CXCR4: T-tropic HIV receptor), expressed on the target cells. Based on these new findings, the manner of entry of HIV into the target cells is currently hypothesized as follows. First, the binding of gp120 and CD4 occurs, and then the resultant binds with CCR5 or CXCR4 expressed on the host cells. As a result, gp120 is structurally altered to expose glycoprotein 41 (gp41), which in turn binds to the cell membrane to establish the infection. On the other hand, corresponding chemokines competitively block the binding of HIV to the chemokine receptors to suppress the HIV infection. The series of findings not only facilitated elucidation of the mechanisms of infection and disease development, but also provided a new viewpoint for anti-HIV strategies.

From such a viewpoint, the present inventors searched metabolites produced by microorganisms, and have found that the culture fluid of a ray fungus strain K97-0003 (deposited on Dec. 9, 1998 to International Patent Organism Depositary (IPOD), National Institute of Advanced Industrial Science and Technology (AIST), Central 6, 1-1 Higashi, Tsukuba, Ibaraki, 305-8566 Japan, under the deposition number FERMBP-6670) has a superior HIV-inhibitory activity and have shown that the active component is a substance that inhibits the syncytium formation of the mechanisms as outlined above (Patent Document 1: International Publication WO11/52043 pamphlet).

Patent Document 1: International Publication WO11/52043 pamphlet

### DISCLOSURE OF THE INVENTION

### Problem to be Solved by the Invention

The present invention is aimed to provide an anti-HIV agent working by inhibiting syncytium formation and having enhanced effects compared to the polypeptide as disclosed in WO0/52043.

### Means for Solving the Problem

The present inventors further investigated for the polypeptide having 114 amino acid residues as disclosed in WO00/52043 (SEQ ID No. 1). As a result, the present inventors have found that a multimer of the polypeptide has greater anti-HIV effects, and completed the present invention.

The present invention provides the following anti-HIV agent(s), polypeptide (s), base sequence (s), transformant (s), and a method for producing the anti-HIV agent(s).

(1) An anti-HIV (human immunodeficiency virus) agent comprising a multimer of actinohivin.
(2) The anti-HIV agent according to (1) above, wherein the multimer of actinohivin is a multimer in which two or more polypeptides selected from:
(a) the polypeptide as defined in SEQ ID No. 1; and
(b) a homologue of (a) above having inhibitory activity on syncytium formation, in which one or more amino acids in the amino acid sequence of (a) have been deleted, substituted or added
   (the polypeptides may be the same or different) are linked with a linker consisting of up to fifty amino acids.
      (3) The anti-HIV agent according to (1) or (2) above, wherein the multimer of actinohivin is a dimer of actinohivin.
      (4) The anti-HIV agent according to any one of (1) to (3) above, wherein the linker is a peptide chain as defined in SEQ ID No. 2.
      (5) The anti-HIV agent according to any one of (1) to (4) above, further comprising a His tag.
      (6) A polypeptide corresponding to the anti-HIV agent according to any one of (1) to (˜5) above.
      (7) A base sequence encoding the polypeptide according to (6) above or a complementary strand thereof.
      (8) A transformant in which the base sequence according to (7) above has been introduced into a host cell.
      (9) The transformant according to (8) above, wherein the host cell is E. coli or actinomycete.
      (10) The transformant according to (9) above, wherein the transformant is Escherichia coli BL21(DE3) pLysS/pET30:dAH strain (International Patent Organism Depositary (IPOD), National Institute of Advanced Industrial Science and Technology (AIST) Accession No. FERM BP-10161).
      (11) A method for producing the anti-HIV agent according to any one of (1) to (5) above, comprising the steps of culturing the transformant according to (9) or (10) above to produce in the transformant cells or in the culture the polypeptide according to (6) above, and isolating the same.

### PREFERRED EMBODIMENTS OF THE INVENTION

### Anti-HIV Agent

In a first embodiment of the invention, an anti-HIV agent comprising a multimer of actinohivin is provided. Actinohivin herein means:

(a) the polypeptide as defined in SEQ ID No. 1; and
(b) a homologue having inhibitory activity on syncytium formation, in which one or more amino acids in the amino acid sequence of (a) above have been deleted, substituted or added.

The polypeptide in which a part of the amino acid sequence has been deleted, substituted or added, as stated in (b) above means a polypeptide having at least 20%, preferably 30% or more, more preferably 50% or more, further more preferably 80% or more, and most preferably 90% or more of amino acid homology with the amino acid sequence as defined in SEQ ID No. 1. As long as the inhibitory activity on syncytium formation is exhibited, the deletion, substitution or addition of an amino acid may occur at any position.

The polypeptide as defined by SEQ ID No. 1 is constituted from three homologous domains (in SEQ ID No. 1, 1-38, 39-76 and 77-114). Those actinohivin multimer (n-mer) with one or two homologous domains added are also included. The number (n) of the actinohivin unit contained in an actinohivin multimer (n-mer) is not restricted as long as the inhibitory activity on syncytium formation is exhibited. Considering the handling in the purification process and so on, normally hexamer or less, preferably trimer or less, e.g., dimer is used.

The inhibitory activity on syncytium formation may be suitably evaluated. For example, evaluation can be conducted based on the presence or absence of fusion of: (α) cells expressing envelope glycoprotein (gp120, gp41) (e.g., HeLa cells) and (β) cells expressing the genes of an auxiliary receptor (CXCR4 or CCR5) of HIV-1 that is essential for the entry of HIV-1 and CD4 (e.g., HeLa cells). In practice, those cells to which a transcription activation protein (Tat) gene has been further introduced and those to which HIV LTR (long terminal repeat sequence) and β-galactosidase gene has been further introduced are used as the (α) and (β), respectively. By using such cells, the presence or absence of syncytium formation (HIV infection) can be judged by color development from X-gal (5-bromo-4-chloro-3-indolyl-beta-galactoside), the substrate for β-galactosidase, because β-galactosidase is activated along with the HIV infection of the cells.

In the context of the present invention, an actinohivin multimer means the amino acid sequences as defined in (a) or (b) above which have been linked each other or one another either directly or via any linker sequence, and may contain a sequence useful for purification or handling of the polypeptide.

The linker sequence is not limited as long as it does not disturb the anti-HIV action of the multimer. It is a sequence consisting of generally fifty or less, preferably 20 or less, more preferably 15 or less amino acid residues. For example, a polypeptide as defined in SEQ ID No. 2 is among those linkers. Accordingly, the actinohivin multimer of the invention includes a polypeptide consisting of the polypeptide of SEQ ID No. 1—the linker sequence of SEQ ID No. 2—the polypeptide of SEQ ID No. 1 (indicated as SEQ ID No. 3). This polypeptide is a dimer. Similar structures of trimer or more are possible.

Examples of those additional sequences useful for the purification or handling of the polypeptide include His tags. GST tags may be used as well, but generally these modification sequences tend to decrease the anti-HIV action. An example of His tags is His₆ tag.

The reason why actinohivin multimers exert excellent activities is not clear. However, it has been demonstrated that actinohivin binds the high-mannose sugar chain of gp120 and thus it is possible that binding as a cluster enhances the anti-HIV effect.

### Polypeptide

In a second embodiment of the present invention, a polypeptide corresponding to said anti-HIV agent comprising an actinohivin multimer is provided. The details of such a polypeptide are similar to those described as the anti-HIV agent.

### Base Sequence

In a third embodiment of the present invention, a base sequence coding for said polypeptide or a complementary strand thereof is provided.

For example, the base sequences provided by the present invention include the base sequence of SEQ ID No. 4 (including a stop codon) corresponding to the polypeptide of SEQ ID No. 3. More generally, base sequences of two or more actinohivin coding regions selected from:

(a′) a base sequence coding for the polypeptide indicated by SEQ ID No. 1; and

(b′) a base sequence coding for a homologue having inhibitory activity on syncytium formation and having an amino acid sequence in which one or more amino acids have been deleted, substituted, or added in the amino acid sequence of (a′),

linked each other via a base sequence coding for a linker sequence, are provided.

As used herein, the meanings of the terms “a homologue”, “a linker sequence”, and “inhibitory activity on syncytium formation” are as defined above. For example, a homologue has at least 20%, preferably 30% or more, more preferably 50% or more, further more preferably 80% or more, and most preferably 90% or more of homology with the base sequence coding for the amino acid sequence as defined in SEQ ID No. 1. As stated above, the actinohivin monomer consists of three homologous domains, and a homologue may contain a base sequence corresponding to one or two homologous domains in addition to a base sequence corresponding to a multimer. The number (n) of actinohivin coding regions contained in a base sequence is not limited as long as the actinohivin multimer eventually obtained retains inhibitory activity on syncytium formation. However, considering the handling during purification and so on as described above, it is normally six or less, preferably three or less, namely two.

In addition, the base sequence of the present invention may contain a sequence useful for the production of the polypeptide. For example, when an actinohivin multimer is expressed in vivo, if it is necessary or beneficial to produce a precursor polypeptide first, and then modify it after translation to obtain the actinohivin multimer as a mature protein, a base sequence for doing so may be contained. Those base sequences include a signal peptide sequence region used for the secretion outside from a microorganism. Further, a sequence useful for the purification or handling of the polypeptide may also be contained. Those base sequences include a base sequence corresponding to a His tag.

In the present application, a base sequence coding for a polypeptide may be coded by any of those degenerate codons. However, base sequences suitable for the expression in a transformant which is described below are preferred. Base sequences may also be a complementary strand of the above described sequences.

### Transformant

In a fourth embodiment of the present invention, a transformant in which the above described base sequence has been introduced into a host cell is provided. The transformant can be anything, as long as it can stably express an actinohivin multimer, and includes E. coli, actinomycete, other bacteria, fungi, yeasts, animal cells, insect cells and so on. Further, cells which have been mutated by cell engineering using techniques such as cell fusion and genetic manipulation are also included in hosts of the transformant of the present invention.

Particularly preferred host cells are E. coli and actinomycete. Examples of those transformants include Escherichia coli BL21 (DE3) pLysS/pET30:dAH strain (deposited on Nov. 10, 2004 with International Patent Organism Depositary (IPOD), National Institute of Advanced Industrial Science and Technology (AIST), Central 6, 1-1 Higashi, Tsukuba, Ibaraki, 305-8566 Japan, under the Accession number FERM BP-10161).

The transformant may be produced using a method known in the relevant art (see, for example, Joseph Sambrook et al., “Molecular Cloning”, 3rd Ed. (Cold Spring Harbor Laboratory Press (2001)).

### Process for Producing the Anti-HIV Agent

In the fourth embodiment of the present invention, a process for producing an actinohivin multimer anti-HIV agent comprising culturing the transformant to cause the production of said polypeptide in the transformant or in the culture and isolating the product.

For culturing the transformant of the present invention, a conventional method normally utilized for culturing such transformant can be adopted. As for the culture medium, either a natural medium or a synthetic medium can be used, as long as it contains a carbon source catabolizable for the transformant, a nitrogen source, an inorganic substance, and so on in proper amounts.

As a carbon source, carbohydrates, such as glucose, mannose, maltose and molasses; organic acids, such as citric acid, malic acid, acetic acidand fumaric acid; alcohols, such as methanol and ethanol; hydrocarbons, such as methane, ethane, propane and n-paraffin; amino acids, such as glutamic acid; or glycerol and so on may be used.

As a nitrogen source, ammonium salts, such as ammonium chloride, ammonium sulfate, ammonium nitrate and ammonium phosphate; amino acids, such as aspartic acid, glutamine, cystine and alanine; urea, peptone, meat extract, yeast extract, dry yeasts, cone steep liquor, soybean flour, soluble vegetable protein, cottonseed oil, soybean casein, casamino acids, Pharmamedia, and so on may be used. As an inorganic substance, potassium monohydrogenphosphate, potassium dihydrogenphosphate, sodium dihydrogenphosphate, magnesium phosphate, magnesium sulfate, ferrous sulfate, manganese sulfate, copper sulfate, cobalt sulfate, zinc sulfate, calcium pantothenate, ammonium molybdate, aluminum potassium sulfate, barium carbonate, calcium carbonate, cobaltous chloride, salt, and so on may be used. In addition, substances for promoting the proliferation of the cells or the production of an actinohivin multimer, such as a small amount of metal salts, vitamins, thiamin, and so on may be added to the medium if needed. Furthermore, when a specific substance is required for the growth of the transformant, it is necessary to add such required substance. These substances may be any of those useful for the production of an actinohivin multimer. For example, all the materials known as being useful for culturing a transformant, particularly E. coli or actinomycetes, can be used.

For large scale culture of a transformant, especially a microorganism, those culture methods that are performed in a liquid medium with shaking, with aerating and stirring, and so on are preferable. The temperature of culturing may be selected within the range in which the transformant can grow and produce an actinohivin multimer. Culturing may be appropriately carried out depending on the nature of the transformant. When the actinohivin multimer is present in the culture fluid, it may be possible to recover the culture fluid containing the cells and to use it per se. However, generally an actinohivin multimer solution is used after the actinohivin multimer and the transformant in the culture fluid have been separated according to a conventional method such as filtration and centrifugation. When the actinohivin multimer is present in the transformant cells, the cells may be recovered from the culture by means of filtration, centrifugation and so on, then disrupted by a mechanical method or an enzymatic method such as the use of lysozyme, and optionally lysed by adding a chelating agent such as EDTA and/or a surfactant when necessary, to separate and obtain the multimer in a form of an aqueous solution.

The solution containing the actinohivin multimer thus obtained can be concentrated by, for example, reduced pressure concentration or membrane concentration, and further precipitated by salting-out using ammonium sulfate or sodium sulfate, or by fractional precipitation using a hydrophilic organic solvent such as methanol, ethanol, acetone, and so on. Then, the precipitate may be solved in water and dialysed with a semi-permeable membrane to remove low molecular weight impurities. Gel filtration with an absorbent or a gel filtration matrix, adsorption chromatography, ion exchange chromatography or reversed phase chromatography may also be utilized for purification. In particular, in the embodiment in which actinohivin multimer containing a His tag is produced, it can be purified by a column with a solid phase having high affinity to the tag, such as Ni-, Zn-, Cu-, or Co-column.

The actinohivin multimer-containing solution obtained by these means can be further subjected to vacuum concentration, lyophilization, and so on to obtain purified actinohivin multimer. The peptide added to the actinohivin multimer, e.g., a His tag, may be further removed as necessary.

### EXAMPLES

The present invention is further explained more specifically below by means of examples. However, these examples are not intended to limit or restrict the scope of the present invention.

### Example 1

### (1) Construction of Gene Coding for Actinohivin Dimer

The gene (SEQ ID No. 4) coding for the actinohivin dimer as defined in SEQ ID No. 3 was constructed according to the protocol as shown in FIG. 1.

Firstly, a linker gene coding for 13 amino acid residues for ligating two actinohivin genes was prepared as follows.

Synthetic oligonucleotide Link-sen (SEQ ID No. 5) and Link-asn (SEQ ID No. 6) were annealed at the homologous sequence sites of 11 bases added to the 3′- and 5′-termini, respectively, of the both primers to form a primer dimer. The resultant was subjected to PCR under the conditions of 30 seconds at 94° C., 60 seconds at 60° C., and 1 minute at 72° C. (30 cycles).

Next, an actinohivin gene corresponding to the N-terminal portion of actinohivin dimer (N-AH gene) was prepared by PCR under the conditions of 30 seconds at 94° C., 30 seconds at 60° C., and 1 minute at 72° C. (30 cycles) using two synthetic oligonucleotide primers AA-1Bam (SEQ ID No. 7) and AH-Link (SEQ ID No. 8), and plasmid 2A3K carrying AH gene cloned from an actinohivin-producing microorganism as the template. The resulting N-AH gene has the addition of 15 base pairs of the 5′-terminal portion of the linker gene at the 3′-terminus of actinohivin gene.

An actinohivin gene corresponding to the C-terminal portion of actinohivin dimer (C-AH gene) was prepared as follows. PCR was conducted under the conditions of 30 seconds at 94° C., 30 seconds at 60° C., and 1 minute at 72° C. (30 cycles) using Link-dAH (SEQ ID No. 9) and TGAEco (SEQ ID No. 10), and plasmid 2A3K as the template. The resulting C-AH gene has the addition of 15 bases of the 3′-terminal portion of the linker gene at the 5′-terminus of actinohivin gene.

L-AH gene, which had the linker gene added at the 5′-terminus of actinohivin gene, was prepared by PCR using the linker gene and C-AH gene as the template and the synthetic oligonucleotides Link-sen and TGAEco as described above, according to the SOE method (Horton, R. M., Hunt, H. D., Ho, S. N., Pullen, J. K. and Pease, L. R. Engineering hybrid genes without the use of restriction enzymes: gene splicing by overlap extension. Gene 77, 61˜68 (1989)).

The thus obtained N-AH gene and L-AH gene were digested with restriction enzymes BamH I and Hind III, and Hind III and EcoR I, respectively. The resulting two DNA fragments were mixed with E. coli cloning vector pUC19 that had been digested with BamH I and EcoRI, and ligated using T4 DNA ligase. E. coli strain JM109 was transformed with the resulting solution to obtain plasmid pUC19:dAH containing actinohivin dimer gene.

### (2) Construction of Actinohivin Dimer Expression Plasmid

An actinohivin dimer gene was amplified by PCR using pUC19:dAH as the template and AA-1 LIC (SEQ ID No. 11) and TGA 114 LIC (SEQ ID No. 12) under the conditions of 30 seconds at 94° C., 30 seconds at 60° C., and 1 minute at 72° C. (30 cycles). Resulting DNA fragments were subjected to T4 DNA polymerase reaction in the presence of dGTP, followed by ligation with E. coli expression vector pET30 Xa/LIC, to construct the actinohivin dimer expression plasmid pET30:dAH.

### (3) Expression of Actinohivin Dimer by E. coli

LA medium used was containing kanamycin sulfate (KM) and chloramphenicol (CM) added so as to make the final concentrations of 30 μg/ml and 34 μg/ml, respectively.

A colony of E. coli BL21(DE3)plyss transformed with the actinohivin dimer expression plasmid pET30:dAH (deposited on Nov. 10, 2004 with International Patent Organism Depositary (IPOD), National Institute of Advanced Industrial Science and Technology (AIST) (Central 6, 1-1 Higashi, Tsukuba, Ibaraki, 305-8566 Japan), under the Accession number FERM BP-10161) formed on LA medium was used to inoculate 1 ml of LB medium containing 2% glucose, KM (30 μg/ml) and CM (34 μg/ml) and cultured at 37° C. overnight with shaking. Eight ml of this culture was added to 800 ml of LB medium containing KM (30 μg/ml) and CM (34 μg/ml) and cultured at 37° C. with shaking. Isopropyl-β-thiogalactopyranoside (IPTG) was added at OD₆₀₀ₙₘ of 0.3, and the culture was continued further two hours at 37° C. with shaking.

After the growth of E. coli was stopped by quenching the medium with iced water, the culture fluid was centrifuged at 12,000×g for 15 minutes at 4° C. to recover the cells. The obtained cells were washed twice with PBS(−) and stored at −20° C.

### Comparable Example 1

### (1) Construction of Actinohivin (Monomer) Expression Plasmid

Actinohivin gene as defined in SEQ ID No. 1 was amplified by performing PCR using plasmid 2A3K as the template and AA-1 LIC (SEQ ID No. 11) and TGA 114 LIC (SEQ IDNo. 12) as described above under the conditions of 30 seconds at 94° C., 30 seconds at 60° C., and 1 minute at 72° C. (30 cycles). The resulting DNA fragment was subjected to T4DNA polymerase reaction in the presence of dGTP and then ligated to E. coli expression vector pET30 Xa/LIC to constract the actinohivin (monomer) expression plasmid pET30:AH.

### (2) Expression of Actinohivin (Monomer) by E. coli

Actinohivin (monomer) was expressed in E. coli BL21 (DE3)plysS transformed by a procedure similar to that in Example 1, (3), except for that the actinohivin (monomer) expression plasmid pET30:AH was used in place of the actinohivin dimer expression plasmid pET30:dAH.

### Example 2

According to the procedures described below, the actinohivin fusion protein was prepared and purified, and tested for its anti-HIV effects.

(1) Preparation of E. coli Extract

According as Example 1, E. coli cells containing the actinohivin dimer with a His tag expressed in the cells were suspended in 10 ml of a binding buffer (5 mM imidazole, 0.5 M NaCl, 20 mM Tris-HCl (pH 7.9)). The cells were disrupted by sonication of 1 minute with 30 seconds interval (total 5 minutes). The resulting E. coli extract was centrifuged at 8,370×g for 10 minutes at 4° C. and the supernatant and pellet were separated. The pellet obtained was dissolved in the binding buffer containing 6 M guanidine (hereinafter, the binding buffer DN), and the solution was centrifuged at 8,370×g for 10 minutes at 4° C. to remove the pellet.

(2) Affinity Chromatography with Metal Chelate Sepharose 4B

Five ml of metal chelate Sepharose 4B was poured in a 15 ml plastic tube, to which 10 ml of milliQ water was added. After gently stirred, the solution was centrifuged at 264×g for 3 minutes at room temperature to remove the supernatant. Five ml of a charge buffer (50 mM NiSO₄) was added and the solution was stirred to activate the resin. After being washed twice with 10 ml of milliQ water, the resin was equilibrated with 10 ml of the binding buffer (DN).

Five ml of activated resin and the sample prepared in (1) above were mixed in a 50 ml plastic tube. After the total volume was adjusted to 50 ml with the binding buffer (DN), the mixture was gently shaken for 1 hour at room temperature. The resin was washed with 10 ml of the binding buffer (DN), then filled in a column, and washed with 50 ml of the binding buffer (DN). Next, the resin was washed with 50 ml of a washing buffer (60 mM imidazole, 0.5 M NaCl, 20 mM Tris-HCl (pH 7.9)) containing 6 M guanidine hydrochloride, and then the His-tagged actinohivin dimer fusion protein adsorbed to the resin was eluted with 50 ml of an elution buffer (500 mM imidazole, 0.5 M NaCl, 20 mM Tris-HCl (pH 7.9)) containing 6 M guanidine hydrochloride.

(3) ODS Column Chromatography

Ten ml of ODS resin suspended in acetonitrile was filled in a column, and the column was washed with 50 ml of 1:1 mixture of acetonitrile: 0.01% trifluoro acetic acid (TFA) and then equilibrated with 50 ml of 5:95 mixture of acetonitrile 0.01% TFA. The His-tagged actinohivin dimer fusion protein purified by metal chelate Sepharose 4B was loaded on the top of the column. After the column was washed with 50 ml of 5:95 mixture of acetonitrile: 0.01% TFA, the His-tagged actinohivin dimer fusion protein adsorbed to the column was eluted with 50 ml of 1:1 mixture of acetonitrile: 0.01% trifluoro acetic acid (TFA). The resulting fluent was concentrated, dried and then dissolved in MilliQ water.

(4) HPLC Using ODS Column

The sample solution desalted with the ODS column was dissolved in a small quantity of MilliQ water, subjected to high performance liquid chromatography (PEGASIL-B, 10φ×250 mm, SSC, Senshu Scientific Co., Ltd.). Using a linear gradient of 5:95 mixture of acetonitrile: 0.01% TFA and 70:30 mixture of acetonitrile: 0.01% TFA in 30 minutes, the peaks appearing at 12 minutes and 16 minutes at the flow rate of 3 ml/min were collected with the absorption at 220 nm being monitored. Each of these fractions were repeatedly frozen and thawed until the pH became around neutral and dissolved in milliQ water.

(5) Measurement of Inhibitory Activity on Syncytium Formation

HeLa/CD4/LTR cells adjusted to 1.6×10⁵ cells/ml with DME medium were dispensed at 50 μl/well into wells of a 96-well plate, to which the sample solution serially diluted with serum-free DME medium was added at 10 μl/well. Further, HeLal35/T-env (envelope glycoprotein derived from T-tropic HIV-1)/Tat cells adjusted to 1.6×10⁵ cells/ml were dispensed at 50 μl/well and the plate was incubated at 37° C. under 5% CO₂ atmosphere for 24 hours. The culture medium was removed and the cell lysis solution was added at 20 μl/well and the plate was kept standing for 15 minutes at room temperature. After 100 μl of chromogenic substrate solution (a mixture of 20 μl of o-nitrophenyl-β-D-glactopyranoside and 80 μl of Z-buffer (60 mM disodium hydrogenphosphate, 40 mM sodium dihydrogenphosphate, 10 mM potassium chloride, 1 mM magnesium sulfate, and 50 mM 2-mercaptoethanol, pH 7.0)) was added to each well and reacted at 37° C. for 80 minutes, the reaction was stopped by adding 25 μl/well of 2 M Na₂CO₃, and the absorbance at 405 nm was measured using PAWER WAVE X340 (BIO TEC INSTROMENTS CO.). The absorbance measurements were used to calculate the percentages of syncytium formation based on the following formula:

The percentage of syncytium formation=(the absorbance of the well with sample addition/the absorbance of the control well)×100 [Formula I]

The concentrations that inhibit 50% of syncytium formation by the actinohivin fusion protein (IC₅₀) thus obtained is shown in Table 1.

Further, by conducting an experiment similar to that described for the preparation of His-tagged the actinohivin dimer fusion protein, the His-tagged actinohivin monomer fusion protein was prepared and the percentage of syncytium formation was measured. The results are also shown in Table 1.

TABLE 1

| | concentration that |
|---|---|
| | inhibit 50% of syncytium |
| His-tagged fusion protein | formation (IC₅₀) |
| actinohivin dimer fusion | 0.235 |
| protein | |
| actinohivin monomer fusion | 0.58 |
| protein | |

Based on the results above, it is demonstrated that the actinohivin dimer fusion protein of the present invention has more than twice inhibitory activity on syncytium formation compared to that of the actinohivin monomer fusion protein.

It is noted that a His tag is known to reduce inhibitory activity on the syncytium formation to about 1/7 to ⅛ and that the concentration that inhibits 50% of syncytium formation of the actinohivin dimer fusion protein without a His tag is expected to be around 0.03 μM.

### INDUSTRIAL APPLICABILITY

The actinohivin multimer protein of the present invention inhibits syncytium formation which is a critical pathway in HIV infection. The working mechanism is considered to be similar to that of the inhibition of syncytium formation by actinohivin and thus it is expected to be effective in preventing infection not only by T-tropic HIV but also by M-tropic HIV. Moreover, the anti-HIV effect of actinohivin dimer protein of the present invention, for example, is more than twice of that of the actinohivin monomer fusion protein, and is remarkable with only a very small amount. Therefore, according to the present invention, an anti-HIV agent polypeptide that can be used as a therapeutic and/or preventive agent of AIDS is provided. Further, the gene coding for the actinohivin multimer protein of the present invention can be introduced into various hosts to produce a recombinant actinohivin multimer protein and it can be readily produced.

### BRIEF DESCRIPTION OF THE DRAWINGS

FIG. 1 is a graphic of the outline of the construction method of the actinohivin dimer.

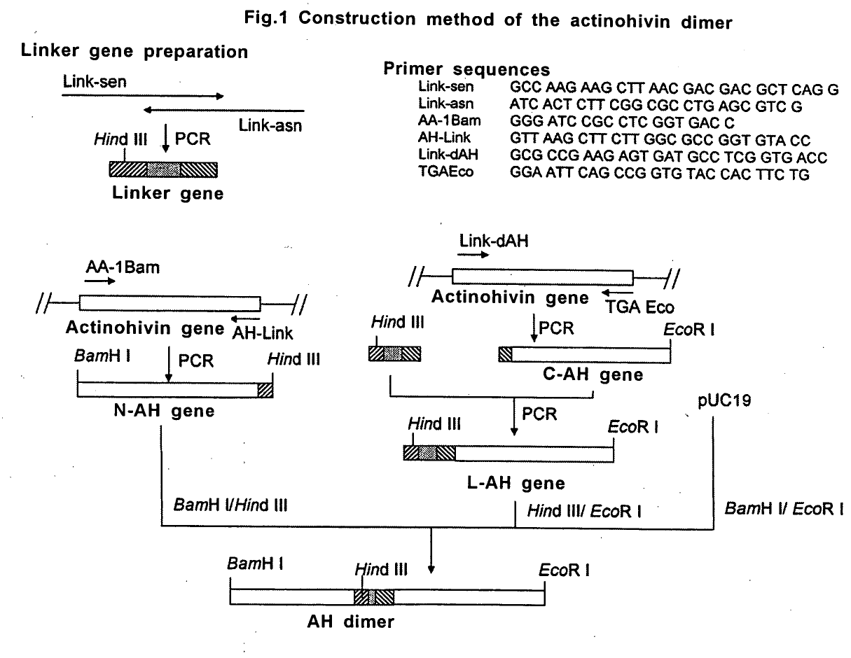

1. An anti-HIV (human immunodeficiency virus) agent comprising a multimer of actinohivin.

2. The anti-HIV agent according to claim 1, wherein the multimer of actinohivin is a multimer in which two or more polypeptides selected from:
(a) the polypeptide as defined in SEQ ID No. 1; and
(b) a homologue of (a) above having inhibitory activity on syncytium formation, in which one or more amino acids in the amino acid sequence of (a) have been deleted, substituted or added
(the polypeptides may be the same or different)
are linked with a linker consisting of up to fifty amino acids.

3. The anti-HIV agent according to claim 1, wherein the multimer of actinohivin is a dimer of actinohivin.

4. The anti-HIV agent according to claim 2, wherein the linker is a peptide chain as defined in SEQ ID No. 2.

5. The anti-HIV agent according to claim 4, wherein the multimer comprises a His tag.

6. A polypeptide corresponding to the anti-HIV agent according to claim 1.

7. A base sequence encoding the polypeptide according to claim 6 or a complementary strand thereof.

8. A transformant in which the base sequence according to claim 7 has been introduced into a host cell.

9. The transformant according to claim 8, wherein the host cell is E. coli or actinomycete.

10. The transformant according to claim 9, wherein the transformant is Escherichia coli BL21 (DE3) pLysS/pET30:dAH strain (International Patent Organism Depositary (IPOD), National Institute of Advanced Industrial Science and Technology (AIST) Accession No. FERM BP-10161).

11. A method for producing the anti-HIV agent according to claim 1, comprising the steps of culturing the transformant according to claim 9 to produce in the transformant cells or in the culture the polypeptide according to claim 6, and isolating the same.


SEQUENCE LISTING (12 sequences)

SEQ ID NO: 1 - protein, 114 aa, Actinomyces K97-0003 Strain
ASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTG

SEQ ID NO: 2 - protein, 13 aa, Artificial sequence
  Linker sequence
AKKLNDAQAPKSD

SEQ ID NO: 3 - protein, 241 aa, Artificial sequence
  Actinohivin dimer
ASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTGAKKLNDAQAPKSDASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTG

SEQ ID NO: 4 - DNA, 726 bp, Artificial sequence
  DNA encoding Actinohivin dimer
gcctcggtgaccatccgcaacgcccagaccggccgcctgctggacagcaactacaacggcaacgtctacacgctgcccgccaacggcgggaactaccagcggtggaccggccccggcgacggcaccgtccgcaacgcccagaccggccgctgcctcgacagcaactacgacggcgccgtctacacgctgccgtgcaacggcggtagctaccagaagtggctgttctacagcaacggctacatccagaacgtcgagaccggacgcgtgctcgacagcaactacaacggcaacgtgtacacactgccggccaacggcggcaactaccagaagtggtacaccggcgccaagaaactgaacgacgctcaggcgccgaagagtgatgcctcggtgaccatccgcaacgcccagaccggccgcctgctggacagcaactacaacggcaacgtctacacgctgcccgccaacggcgggaactaccagcggtggaccggccccggcgacggcaccgtccgcaacgcccagaccggccgctgcctcgacagcaactacgacggcgccgtctacacgctgccgtgcaacggcggtagctaccagaagtggctgttctacagcaacggctacatccagaacgtcgagaccggacgcgtgctcgacagcaactacaacggcaacgtgtacacactgccggccaacggcggcaactaccagaagtggtacaccggctga

SEQ ID NO: 5 - DNA, 28 bp, Artificial sequence
  Primer Link-sen
gccaagaagcttaacgacgacgctcagg

SEQ ID NO: 6 - DNA, 25 bp, Artificial sequence
  Primer Link-asn
atcactcttcggcgcctgagcgtcg

SEQ ID NO: 7 - DNA, 19 bp, Artificial sequence
  Primer AA-1Bam
gggatccgcctcggtgacc

SEQ ID NO: 8 - DNA, 26 bp, Artificial sequence
  Primer AH-Link
gttaagcttcttggcgccggtgtacc

SEQ ID NO: 9 - DNA, 27 bp, Artificial sequence
  Primer Link-dAH
gcgccgaagagtgatgcctcggtgacc

SEQ ID NO: 10 - DNA, 26 bp, Artificial sequence
  Primer TGAEco
ggaattcagccggtgtaccacttctg

SEQ ID NO: 11 - DNA, 30 bp, Artificial sequence
  Primer AA-1 LIC
ggtattgagggtcgcgcctcggtgaccatc

SEQ ID NO: 12 - DNA, 30 bp, Artificial sequence
  Primer TGA 114 LIC
agaggagagttagagcctcagccggtgtac